(19) United States
(12) Patent Application Publication
(10) Pub. No.: US 2026/0083676 A1
(43) Pub. Date: Mar. 26, 2026

(54) SPRAY-DRIED AMORPHOUS SOLID DISPERSIONS AND METHOD FOR PREPARATION

(71) Applicant: Merck Patent GMbH, Darmstadt (DE)

(72) Inventors: Lena Mueller, Schluechtern (DE); Laura Halstenberg, Rodgau (DE); Thomas Kipping, Darmstadt (DE); Nicole Di Gallo, Bensheim (DE)

(21) Appl. No.: 19/110,241

(22) PCT Filed: Sep. 14, 2023
(86) PCT No.: PCT/EP2023/075219

§ 371 Date: Mar. 10, 2025

(30) Foreign Application Priority Data
Sep. 16, 2022 (EP) 22196015.6

(51) Int. Cl. A61K 9/16; A61K 31/405; A61K 31/427; A61K 31/454; A61K 31/496; A61K 31/501; A61K 31/551

(52) U.S. Cl. CPC A61K 9/1641; A61K 9/1694; A61K 31/405; A61K 31/427; A61K 31/454; A61K 31/496; A61K 31/501; A61K 31/551

(57) ABSTRACT

The present invention relates to the use of polyvinyl alcohol for spray-drying to form an amorphous solid dispersion of an active pharmaceutical ingredient in a polyvinyl alcohol matrix. Furthermore, the invention relates to a process for producing an amorphous solid dispersion of an active pharmaceutical ingredient in a polyvinyl alcohol matrix by spray-drying.

### TECHNICAL FIELD

The present invention relates to the use of polyvinyl alcohol for spray-drying to form an amorphous solid dispersion of an active pharmaceutical ingredient in a polyvinyl alcohol matrix. Furthermore, the invention relates to a process for producing an amorphous solid dispersion of an active pharmaceutical ingredient in a polyvinyl alcohol matrix by spray-drying.

### BACKGROUND

Formulation of a drug product needs to be carefully designed when the Active Pharmaceutical Ingredient (API) exhibits poor solubility and/or poor bioavailability. The Biopharmaceutical Classification System (BCS) arranges these APIs in class II (poor solubility) and class IV (poor solubility and poor bioavailability). They pose a challenge in the dissolution from the final formulation and with 40% of marketed products and 70-90% of New Chemical Entities (NCE) classified in class II and IV of the BCS this challenge affects the majority of formulation work. The greater lipophilicity, higher molecular weights and the resulting poor water solubility derive from rising trends in combinatorial chemistry and new drug design.

Poor aqueous solubility is often linked to the stable, crystalline state of the API. This state can be overcome by disruption of the crystalline lattice and the manufacturing of the amorphous state. This state displays a higher energy form but is less stable than the crystalline counterpart of the same API. Due to this higher energy state, the amorphous form leads to an increased apparent solubility that results in a supersaturated state and improved bioavailability in the Gastrointestinal Tract (GIT). This behavior is described in a spring-parachute analogy: the extra free energy and increased dissolution is known as the spring whereas the parachute helps in slowing down the decrease of free dissolved drug. The parachute is an auxiliary material that additionally helps in stabilizing the high energy amorphous state of the API, prolongs storage and facilitates handling and manufacturing. This material is known as the carrier and can be e.g. a polymer. The polymer reduces the molecular mobility of the API, increases the glass transition temperature, protects the API from re-crystallization and can also facilitate wetting during dissolution if the polymer itself is water soluble.

To generate these Polymeric Amorphous Solid Dispersions (PASD/ASD), methods that involve 1) melting, 2) solvent evaporation and 3) melting-solvent evaporation can be applied (Kim et al. 2021). Spay Drying (SD) can be described as a solvent evaporation method as the liquid feedstock in which API and polymer are dissolved, is forwarded and sprayed into a drying chamber to produce dried particulates that can be then collected. This technology is the method of choice for heat sensitive materials as the cooling effect during vaporization of the liquid protects the particulate during the drying process which, in addition, only takes milliseconds. Advantages of this rapid drying is the well-mixed drug polymer system in the product. SD can be applied in all development stages: from discovery to development and can later be scaled-up to commercial processes. In marketed drug products, spray-drying and hot melt extrusion are the method of choice and account for the majority of registered products (Bhujbal, Mitra, et al. 2021).

To generate droplets from the feedstock, the liquid is transported through a nozzle. For pharmaceutical applications, pneumatic nozzles are mainly used. The liquid is atomized by a stream of gas that can be either air or an inert gas of choice (Ziaee et al. 2019). The droplets are generated at the tip of the nozzle where the liquid stream is broken by the gas stream. These nozzles are multi-fluid with one fixed gas channel and either one or more fluid channels. For pharmaceutical applications in which the components are not co-soluble in the same solvent system, a three-fluid nozzle makes spray-drying possible due to the availability of two fluid channels. This can be used in co-spray-drying of two APIs (Focaroli et al. 2020) or in ASDs in which the polymer and the API need different solvent systems (Bhujbal, Su, et al. 2021).

In the standard spray-drying process, the ingredients of choice are usually dissolved or dispersed in a common solvent that acts as the feed solution or feed suspension. The feedstock is then forwarded to a two-fluid nozzle where one channel holds the liquid and the other the pressurized gas (N2 or air). The need of the amorphous form of an API is usually based on the poor water solubility, therefore the API is dissolved in an organic solvent. To generate the ASD, the polymer is dissolved in the same solvent and spray-dried to yield the desired product. Only when the API is completely dissolved, the amorphous state can be generated, a dispersion or suspension leads to an unstable or crystalline product (Bhujbal, Mitra, et al. 2021).

There is a pool of polymers available that are soluble in organic solvents but a co-solubility of API and polymer is not always feasible. For such challenging combinations, the set-up using a three-fluid nozzle can be the solution in where an additional channel is available that forwards a second liquid stream. This can be applicable in the ASD generation using an API (Naproxen) and polymer (PVP) (Bhujbal, Su, et al. 2021) or in the development of a co-spray-dried formulation for pulmonary delivery of theophylline and salbutamol sulfate (Focaroli et al. 2020).

The use of hydrophilic polymers such as PVA as excipient for pharmaceutical compositions has been widely described. WO 2018/083285 A1 discloses powdered PVA having improved properties as a polymer matrix in pharmaceutical compositions comprising active ingredients, especially in compressed tablets forming amorphous solid dispersions with poorly soluble active pharmaceutical ingredients (APIs). ASDs with PVA as a polymer are known to significantly increase the solubility of poorly water-soluble drugs (Brough et al. 2016). While the preparation of ASDs comprising PVA by melting (e.g. melt or hot-melt extrusion) is well described, there is little to no experience concerning the preparation of ASDs comprising PVA by the method of spray-drying. The only publication that made use of PVA in a spray-drying application was published in 2011 and describes the application of PVA 22,000 and Celecoxib in a suspension that is fed through a two-fluid nozzle (Fouad et al. 2011). One disadvantage is that not all generated products are fully amorphous in the XRD rendering those compositions improper for solubility enhancement and pharmaceutical use. Additionally, polyvinyl alcohol is not listed as a polymer suitable for ASD generation (Hugo, Kunath, and Dressman 2013) and an additional recent review excludes PVA from a list of polymers for spray-dried dispersions (Bhujbal, Mitra, et al. 2021).

To guarantee a stable, fully amorphous ASD formulation, complete dissolution of the API is one of the critical steps. PVA is only soluble in water (or water-ethanol/water-methanol systems) whereas the poorly soluble APIs usually request organic solvents. To generate a stable ASD using PVA, co-dissolving is therefore a challenge and not feasible.

By creating an amorphous solid dispersion usually both ingredients, the drug substance and the polymer are dissolved in a common solvent. This is often a limitation for water based polymers like PVA and the reason why they are not considered as suitable for spray-drying due to their limited solubility in organic solvents. This often excludes the application of a common solvent of polymer and low-soluble drug substances. Additionally, the PVA grade defines the performance in solubility enhancement of poorly soluble APIs therefore not every PVA grade is applicable. Also the water solubility itself is strongly dependent on the hydrolysis degree and the molecular weight of the respective PVA grade.

Currently spray-drying with PVA is rarely applied in pharmaceutical processes as the classical PVA grades show a limited solubility in common solvents or solvent mixtures.

Therefore, there is a need for a method of preparation of a spray-dried ASD with PVA as a polymer. A further object of the present invention is to find optimal PVA grades and/or ranges for PVA grades, suitable for spray-drying to manufacture an ASD. Additionally, a combination of (i) optimal PVA grades and/or ranges for PVA grades and (ii) a spray-drying technique is needed to improve the properties of resulting ASDs. Particular properties to improve are the degree of amorphization of the API in the ASD, the dissolution behaviour of the API and/or the stability of the amorphous form of the API in the ASD.

### SUMMARY OF THE INVENTION

Surprisingly it was found that a PVA having a hydrolysis degree of 90% or lower is especially suitable for the preparation of an ASD using spray-drying, in particular three-fluid spray-drying, as it leads to an improved amorphization, dissolution and/or stability of the API.

In dissolving the API and PVA in two different solvents and using a set-up with a three-fluid nozzle the issue of co-solving is targeted, especially when the desired drug loading cannot be achieved in a two-fluid nozzle set-up. Additionally, the PVA grade needs to be carefully evaluated. While high viscosity values lead to fast clogging of the nozzle, a high hydrolysis grade may lead to solubility issues of the PVA.

In a further embodiment of the invention, the PVA has a hydrolysis degree of between 74% to 88% and a viscosity of a 4% solution at 20° C. of between 3 mPas to 5 mPas, in particular the PVA is PVA 3-82, 4-88 or 5-74.

A further embodiment of the invention is a process for producing an amorphous solid dispersion of at least one active pharmaceutical ingredient in a polyvinyl alcohol matrix by three-fluid nozzle spray-drying, comprising the steps of
a) preparing a first feed solution comprising the at least one active pharmaceutical ingredient,
b) preparing a second feed solution comprising a polyvinyl alcohol having a hydrolysis degree of 90% or lower,
c) spray-drying the first and second feed solution with a three-fluid nozzle, preferably wherein the first feed solution is the inner phase and the second feed solution is the outer phase in the three-fluid nozzle.

In another aspect, the invention provides a process for producing an amorphous solid dispersion of at least one active pharmaceutical ingredient in a polyvinyl alcohol matrix by spray-drying, comprising the steps of
a) preparing a feed solution comprising the at least one active pharmaceutical ingredient and at least one polyvinyl alcohol having a hydrolysis degree of 90% or lower,
b) spray-drying the feed solution.

A further aspect of the invention concerns an amorphous solid dispersion of at least one active pharmaceutical ingredient in a polyvinyl alcohol matrix obtainable by a process as defined above.

A further aspect of the invention concerns a pharmaceutical dosage form comprising an amorphous solid dispersion of at least one active pharmaceutical ingredient in a polyvinyl alcohol matrix obtainable by a process as defined above.

### DETAILED DESCRIPTION OF THE INVENTION

An embodiment of the invention is the use of polyvinyl alcohol in a process for spray-drying to form an amorphous solid dispersion of the at least one active pharmaceutical ingredient in a polyvinyl alcohol matrix, wherein the polyvinyl alcohol has a hydrolysis degree of 90% or lower.

In a particular embodiment the spray-drying is a three-fluid nozzle spray-drying.

Spray-drying is a process of converting a liquid into a dried state. The applied solvent can be aqueous or any organic solvent or mixtures thereof. The liquid form can be a solution, suspension, dispersion, emulsion or paste, whereas the resulting dry product can be a powder, agglomerates or granulars with various particle morphologies and particle sizes. Possible resulting particle morphologies are irregular particles, spheres or satellites, hollow spheres and cenospheres, solid particles, shriveled particles, or encapsulated products. Particle sizes can range from 100 nm to 1000 μm. Spray-drying is applied for drying of a liquid, for micronization, encapsulation of liquids and solids, agglomerations, granulation and to generate amorphous solid dispersions.

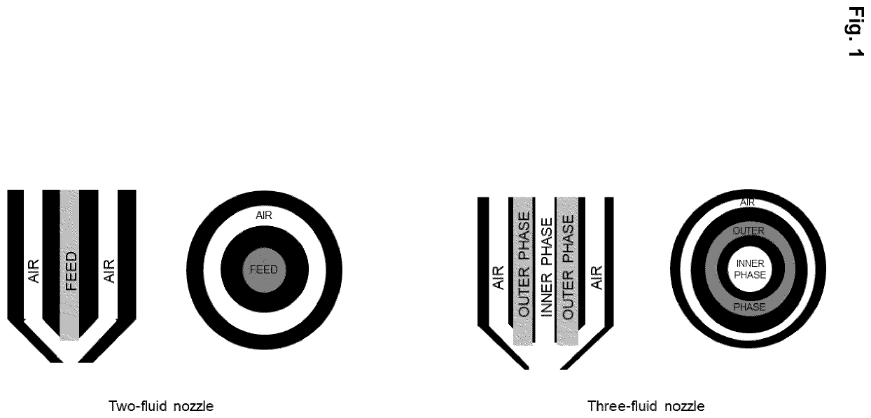

During the drying process, the liquid feed is transported through a nozzle which generates fine droplets that are subsequently sprayed into a heated drying chamber. Commonly applied nozzles include rotary atomizers, pressure nozzles, ultrasonic nozzles and two-fluid as well as three-fluid nozzles with various diameters. Furthermore, inkjet, aerosol-assisted and electrostatic atomizers are possible nozzle types. A schematic illustration of a two-fluid nozzle and three-fluid nozzle can be seen in FIG. 1.

In the heated drying chamber the solvent system is removed completely by evaporation. The drying medium can be air or inert gases that flows through the spray-drying system in a uniform manner. The flow of drying gas can be in co-current, counter-current to the nozzle direction or in a mixed mode.

To further separate the dried particles from the drying gas a separation device is used. For this purpose, a cyclone, bag filters, wet collectors or electrostatic precipitators can be applied.

The two-fluid nozzle is a common set-up where the liquid and the gas come in contact at the exit of the nozzle or internally before exiting into the spraying zone. In both cases there is a dedicated channel for the atomization air and one additional channel for the liquid that is transported into the nozzle. For this process, a peristaltic pump can be applied. In the following this method is referred to as “two-fluid nozzle spray-drying”.

To feed two liquids independently, a three-fluid nozzle can be applied. This nozzle includes a third channel for an additional liquid feed. Using this set-up, divers liquids, e.g. inmiscible systems can be co-spray-dried, or encapsulation facilitated. In the following this method is referred to as “three-fluid nozzle spray-drying”. According to the invention, a three-fluid nozzle has two channels for the liquid phases, an inner phase (inner channel) and an outer phase (outer channel), and one surrounding channel for the atomization air. (Bhujbal, Su, et al. 2021).

Polyvinyl alcohol (PVA) is a synthetic water-soluble polymer that has the idealized formula [CH₂CH(OH)]ₙ. It possesses good film-forming, adhesive, and emulsifying properties. PVA is prepared from polyvinyl acetate, where the functional acetate groups are either partially or completely hydrolysed to alcohol functional groups. If not completely hydrolysed, PVA is a random copolymer consisting of vinyl alcohol repeat units —[CH₂CH(OH)]— and vinyl acetate repeat units —[CH₂CH(OOCCH₃)]—. The polarity of PVA is closely linked to its molecular structure. The hydrolysis degree and the molecular weight determine the molecular properties of PVA. As the degree of hydrolysis of acetate groups increases, the solubility of the polymer in aqueous media and also crystallinity and melting temperature of the polymer increase. However, at high hydrolysis degrees over 88%, the solubility of PVA decreases again. PVA is generally soluble in water, but almost insoluble in almost all organic solvents, excluding, in some cases, ethanol.

The typical PVA nomenclature indicates the viscosity of a 4% solution at 20° C. and the degree of hydrolysis of the polymer. For example, PVA 3-83 is a PVA grade with a viscosity of 3 mPas that is 83% hydrolysed, i.e. having 83% of vinyl alcohol repeat units and 17% of vinyl acetate repeat units. A skilled person is aware that a hydrolysis grade of 83% and a viscosity of 3 mPas encompasses calculated hydrolysis grades of 82.50% to 83.49% and calculated viscosities of 2.50 mPas to 3.49 mPas according to common rounding methods. Viscosity according to the invention is measured as stated in USP 39 under Monograph “Polyvinyl Alcohol” with the method Viscosity-Rotational Method (912).

The degree of hydrolysis according to the invention is measured by determining the saponification value of the Polyvinyl Alcohol, e.g. as stated in USP 39 under Monograph “Polyvinyl Alcohol” under “Degree of Hydrolysis”:

### Sample: 1 g of Polyvinyl Alcohol, Previously Dried at 110° to Constant Weight Analysis:

Transfer the Sample to a wide-mouth, 250-ml conical flask fitted by means of a suitable glass joint to a reflux condenser. Add 35 ml of dilute methanol (3 in 5) and mix gently to ensure complete wetting of the solid. Add 3 drops of phenolphthalein TS, and add 0.2 N hydrochloric acid or 0.2 N sodium hydroxide if necessary, to neutralize. Add 25.0 ml of 0.2 N sodium hydroxide VS, and reflux gently on a hot plate for 1 h. Wash the condenser with 10 ml of water, collecting the washings in the flask, cool, and titrate with 0.2 N hydrochloric acid VS. Concomitantly perform a blank determination in the same manner, using the same quantity of 0.2 N sodium hydroxide VS.

### Calculation of Saponification Value:

### Calculate the Saponification Value:

$$Result = [ ( VB - VS ) \times N \times Mr ] / W$$

VB=volume of 0.2 N hydrochloric acid VS consumed in the titration of the blank (ml)
VS=volume of 0.2 N hydrochloric acid VS consumed in the titration of the Sample solution (ml)
N=actual normality of hydrochloric acid VS
Mr=molecular weight of potassium hydroxide, 56.11
W=weight of the portion of Polyvinyl Alcohol taken (g)

### Calculation of Degree of Hydrolysis:

### Calculate the Degree of Hydrolysis, Expressed as a Percentage of Hydrolysis of Polyvinyl Acetate:

$$Result = 100 - [ 7.84 \times S / ( 100 - 0.075 \times S ) )$$

S=saponification value of the Polyvinyl Alcohol

According to the present invention the PVA grade PVA 3-82 refers to a PVA with the following specifications:
pH: 5.0-6.5
Viscosity: 2.55-3.45
Ester value: 180-220

The ester value I_{E} according to Ph.Eur. 10.8 is the number that expresses in milligrams the quantity of potassium hydroxide required to saponify the esters present in 1 g of the substance. It is calculated from the saponification value I_{S} and the acid value I_{A}:

$${I}_{E} = {I}_{S} - {I}_{A}$$

The use of PVA grades according to the invention is of interest for the formulation of solid oral pharmaceutical dosage forms with an instant, immediate or prolonged API release.

Preferred PVAs have a hydrolysis degree of between 70% to 90%, more preferably between 74% to 88%. With regard to the viscosity of the PVA grades for the use according to the invention, in principle all PVA grades with a viscosity suitable for spray-drying are applicable for the spray-drying methods according to the invention. The skilled person in the art knows to select a PVA grade with a suitable viscosity for those methods. In a preferred embodiment the PVAs have a viscosity of a 4% solution at 20° C. of 20 mPas or lower, in a further preferred embodiment the PVAs have a viscosity of a 4% solution at 20° C. of between 1 to 18 mPas, in a further preferred embodiment the PVAs have a viscosity of a 4% solution at 20° C. of between 2 to 10 mPas, more preferably a viscosity of a 4% solution at 20° C. of between 2 to 5 mPas, most preferably a viscosity of a 4% solution at 20° C. of between 3 to 5 mPas.

In one embodiment the polyvinyl alcohol has a hydrolysis degree of between 70% to 90% and a viscosity of a 4% solution at 20° C. of between 1 to 18 mPas. In a preferred embodiment the polyvinyl alcohol has a hydrolysis degree of between 70% to 90% and a viscosity of a 4% solution at 20° C. of between 2 to 10 mPas. In a more preferred embodiment the polyvinyl alcohol has a hydrolysis degree of between 74% to 88% and a viscosity of a 4% solution at 20° C. of between 2 to 5 mPas. In a most preferred embodiment the polyvinyl alcohol has a hydrolysis degree of between 74% to 88% and a viscosity of a 4% solution at 20° C. of between 3 to 5 mPas.

Preferably, the polyvinyl alcohol is PVA 3-80, PVA 3-81, PVA 3-82 PVA 3-83, PVA 2-88, PVA 3-88, PVA 4-88, PVA 5-88, PVA 2-74, PVA 3-74, PVA 4-74 or PVA 5-74, more preferably PVA 3-82, 4-88 or 5-74, most preferably PVA 3-82.

The above mentioned PVAs, PVA specifications and PVA grades apply equally for (i) the use of polyvinyl alcohol in a process for spray-drying and three-fluid nozzle spray-drying, (ii) the process for producing an amorphous solid dispersion of at least one active pharmaceutical ingredient in a polyvinyl alcohol matrix by spray-drying and three-fluid spray-drying, (iii) the amorphous solid dispersion of at least one active pharmaceutical ingredient in a polyvinyl alcohol matrix obtainable by such processes and (iv) the pharmaceutical dosage form comprising an amorphous solid dispersion of at least one active pharmaceutical ingredient in a polyvinyl alcohol matrix obtainable by such a processes.

The active pharmaceutical ingredient (API) is a biologically active agent. The API may be a small molecule in form of a weak base, a weak acid or a neutral molecule and may be in the form of one or more pharmaceutically acceptable salts, esters, derivatives, analogues, prodrugs, and solvates thereof. The ASD of the present invention may comprise more than one API. In one embodiment the API is poorly soluble or a lipophilic API.

As used herein, the terms “poorly soluble API”, “poorly water-soluble API” and “lipophilic API” refer to an API having a solubility such that the highest therapeutic dose of the particular API to be administered to an individual cannot be dissolved in 250 ml of aqueous media ranging in pH from 1 to 8 following the definition of low solubility according to the Biopharmaceutics Classification System (BCS) classes 2 and 4. Poorly soluble APIs with weakly basic or weakly acidic characteristics have a pH-dependent solubility profile and can have a wide range of solubility in the aqueous environment of the gastrointestinal tract. APIs falling under BCS classes 2 or 4, respectively, are well known to persons skilled in the art.

In one embodiment the API is a weakly basic API. As used herein, the term “weakly basic API” refers to a basic active pharmaceutical ingredient (API) wherein the basic API does not completely ionize in water.

In a preferred embodiment, the API is a PROTAC. According to the invention, a “PROTAC” or “proteolysis targeting chimera” is a heterobifunctional molecule composed of two active domains and a linker, capable of removing specific unwanted proteins. Compared to conventional enzyme inhibitors, PROTACs work by inducing selective intracellular proteolysis consisting of two covalently linked protein-binding molecules: one capable of engaging an E3 ubiquitin ligase, and another that binds to a target protein meant for degradation. Recruitment of the E3 ligase to the target protein results in ubiquitination and subsequent degradation of the target protein via the proteasome.

The API included in the pharmaceutical dosage form of the present invention has a sufficient amount to be therapeutically effective. For a given API, therapeutically effective amounts are generally known or readily accessible by persons skilled in the art. Typically, the API may be present in the pharmaceutical dosage form in a weight ratio of API to polyvinyl alcohol of 0.1:99.1 to 60:40, preferably 1:99 to 50:50, more preferably 5:95 to 40:60 and most preferably 10:90 to 30:70.

According to the invention, an amorphous solid dispersion of at least one active pharmaceutical ingredient is formed. As used herein, the term “amorphous solid dispersion” or “ASD” is a dispersion of at least one amorphous API in a PVA matrix. Preferably, the amorphous API is distributed in a molecularly dispersed state within the polymer matrix. In this case, the solid dispersion is a solid solution. ASDs according to the invention are showing no significant diffraction peaks of crystalline API in a X-ray powder diffraction (XRD) measurement and no melting peak of the API in a differential scanning calorimetry (DSC) measurement. Upon dissolution, formulations comprising an amorphous solid dispersion can reach higher solubilities in aqueous media than the crystalline API.

A further embodiment of the invention is a process for producing an amorphous solid dispersion of at least one active pharmaceutical ingredient in a polyvinyl alcohol matrix by spray-drying, comprising the steps of
a) preparing a feed solution comprising the at least one active pharmaceutical ingredient and at least one polyvinyl alcohol having a hydrolysis degree of 90% or lower,
b) spray-drying the feed solution.

The spray-drying can be a two-fluid nozzle spray-drying or a three-fluid nozzle spray-drying.

In a further embodiment it is a process for producing an amorphous solid dispersion of at least one active pharmaceutical ingredient in a polyvinyl alcohol matrix by three-fluid nozzle spray-drying, comprising the steps of
a) preparing a first feed solution comprising the at least one active pharmaceutical ingredient,
b) preparing a second feed solution comprising a polyvinyl alcohol having a hydrolysis degree of 90% or lower,
c) spray-drying the first and second feed solution with a three-fluid nozzle.

Preferably, the first feed solution is the inner phase and the second feed solution is the outer phase in the three-fluid nozzle.

Feed solutions are typically prepared by dissolving the required amount of PVA in water. Preferably, deionized water is used. Preferably, the solution is heated to a temperature of 40° C. or higher. The respective API is dissolved in a suitable solvent under stirring until a solution is obtained. For the two-fluid nozzle set-up the API solution is added to the PVA solution. Alternatively, the feed solution for the two-fluid nozzle set-up can be prepared in one step by directly dissolving the API and PVA in a suitable solvent.

Suitable solvents for preparing the feed solutions are commonly used solvents for pharmaceutical and spray-drying applications which are known to the skilled person in the art, e.g. water, methanol, ethanol, aceton or mixtures thereof.

In a further embodiment, the process can comprise additional steps such as feeding one or both solutions or suspensions using pneumatic pumps into a two-fluid or three-fluid nozzle. The respective nozzle sprays the liquid into a spraying chamber with elevated temperature where particles are dried and collected with a separating device.

For the avoidance of doubt, the processes according to the invention include two-fluid nozzle spray-drying and three-fluid nozzle spray-drying with any of the PVAs, PVA specifications or PVA grades as defined above.

A further aspect of the invention concerns an amorphous solid dispersion of at least one active pharmaceutical ingredient in a polyvinyl alcohol matrix obtainable by a process as defined above.

A further aspect of the invention concerns a pharmaceutical dosage form comprising an amorphous solid dispersion of at least one active pharmaceutical ingredient in a polyvinyl alcohol matrix obtainable by a process as defined above.

For the avoidance of doubt, the amorphous solid dispersions and pharmaceutical dosage forms according to the invention can include any of the PVAs, PVA specifications or PVA grades as defined above.

It was surprisingly found that there is an optimum of PVA grades or ranges of PVA grades with certain specifications that can be beneficially used in a process of spray-drying to form an amorphous solid dispersion of at least one active pharmaceutical ingredient in a polyvinyl alcohol matrix.

It was found that only PVA grades with a hydrolysis degree of 90% or lower resulted in spray-dried ASDs with a suitable amorphization grade and/or dissolution behavior. The viscosity of the PVA is of minor importance for the ASD formation unless the viscosity is low enough to be used in a spray-drying process. Suitable viscosity of PVA for a spray-drying process are known to the skilled person in the art.

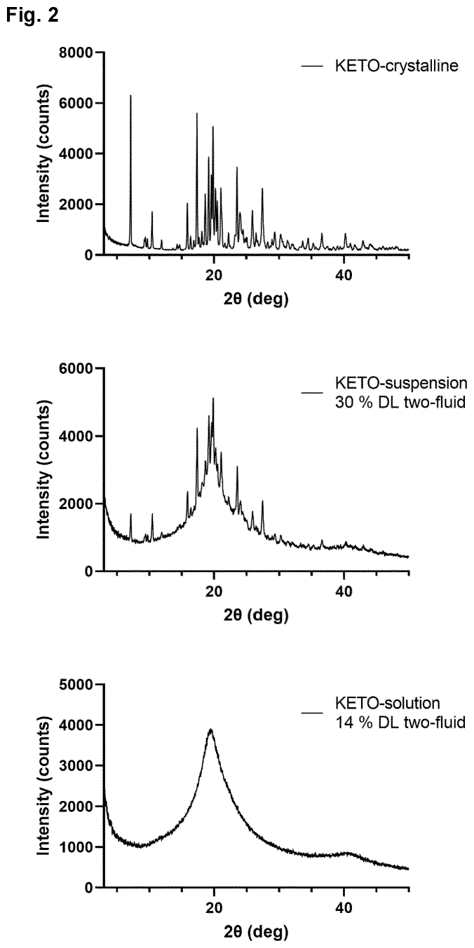

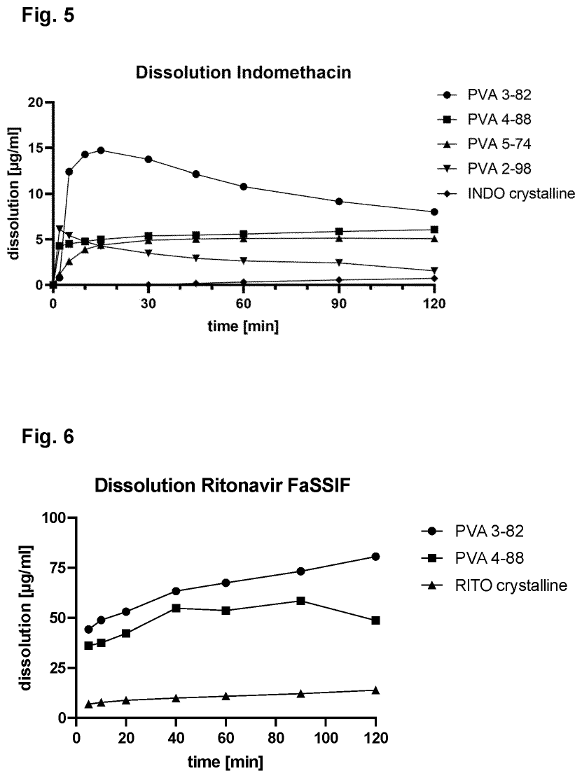

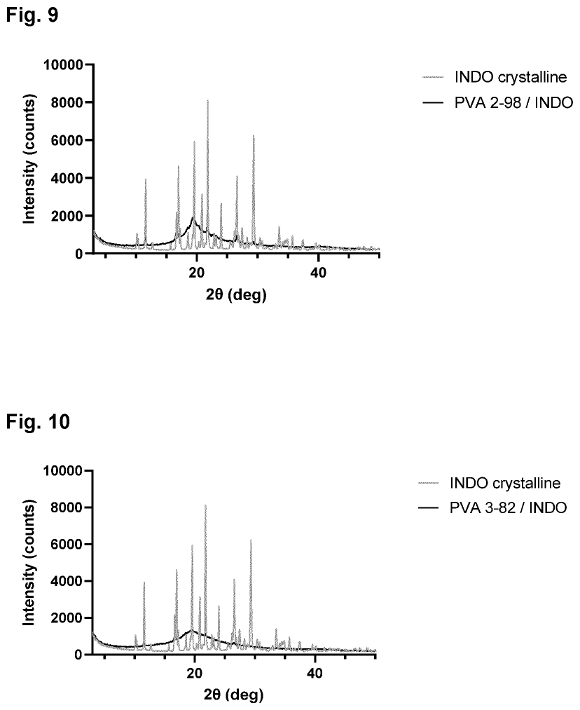

At a certain API-loading of the PVA matrix, a three-fluid nozzle spray-drying leads to an amorphous product whereas a commonly used two-fluid nozzle leads to a partly crystalline product (FIG. 2-FIG. 4). It was surprisingly found that not all PVA grades are suitable for that particular method as PVA 4-98 and PVA 3-98 showed dissolving issues when preparing spray solutions. Furthermore, other PVA grades did not lead to the expected amorphization grade (PVA 2-98, FIG. 9) and also resulted in low dissolution levels (FIG. 5). Surprisingly only PVA grades with a hydrolysis degree of 90% or lower resulted in ASDs with a suitable amorphization grade and dissolution behavior. PVA grades PVA 3-82, 4-88 and 5-74 showed the best results in both amorphization grade and dissolution. Unexpectedly, PVA 3-82 showed superior dissolution profiles compared to all other PVA grades. Additionally, PVA grade PVA 3-82 was able to stabilize the amorphous form of the API in the ASD formulation during storage at different conditions and no significant reduction in dissolution enhancement could be seen.

Furthermore, PVA having the above-identified hydrolysis degree and/or viscosities assure and stabilize the release and supersaturation of APIs, in particular poorly soluble APIs, in aqueous media thereby preventing crystallization and phase separation. Since a low water solubility of an API in general accompanies a low bioavailability after its administration in a pharmaceutical preparation, the compositions according to the invention also contribute to improving the bioavailability of poorly water-soluble APIs, and particularly weakly basic APIs

As used herein, “bioavailability” is a term meaning the degree to which an API becomes available to the target tissue after being administered to the body of a patient.

After dissolving pharmaceutical dosage forms of the present invention an improved supersaturation is observed and the API is kept better in solution. Having entered the gastrointestinal tract, the pharmaceutical dosage form swells and disintegrates in the aqueous environment of the gastrointestinal fluids thereby releasing the API. While a salt form of a weakly basic API may show improved initial aqueous concentration in the acidic gastric fluid, the weakly basic API rapidly converts to the free base form in the more neutral intestinal fluid where the free base-form of the API has a significantly lower equilibrium concentration. PVAs according to the invention maintain enhanced concentrations of the API in model solutions simulating acidic and neutral gastrointestinal solutions as compared to PVAs falling outside the mentioned specifications. Therefore, the pharmaceutical dosage forms according to the invention have the potential to provide enhanced bioavailability of poor solubility APIs. The solubility-improved form of the API in the presence of a PVA according to the invention provides a concentration of the API in gastric fluid or simulated gastric fluid that is greater than the concentration of the API provided in the presence of a PVA falling outside the mentioned specifications.

### EXAMPLES

### Example 1: Two-Fluid Nozzle Spray-Drying Vs Three-Fluid Nozzle Spray-Drying of Solutions and Suspensions

Properties of products prepared by (i) two-fluid nozzle spray-drying and (ii) three-fluid nozzle spray-drying were compared using PVA grade PVA 4-88 and ketoconazole (KETO) as API in solution and suspension

### Spray-Drying

The following feed solutions and suspensions (Table 1) were spray-dried using the Büchi spray-dryer B-290 (BÜCHI, Switzerland) equipped with a high-performance cyclone and the Büchi Inert Loop B-295 (BÜCHI, Switzerland), to inert the system with nitrogen.

TABLE 1

| Formulation | PVA | Ketoconazole | | |
|---|---|---|---|---|
| No./ | content | drug loading | Nozzle | Solvent |
| Description | and grade | (DL) | type | system |
| 1. KETO- | 70% PVA | 30% | Two-fluid | 1:1 |
| Suspension | 4-88 | | nozzle | water:ethanol |
| 30% DL; two- | | | | |
| fluid | | | | |
| 2. KETO- | 86% PVA | 14% | Two-fluid | 1:1 |
| Solution 14% | 4-88 | | nozzle | water:ethanol |
| DL; two-fluid | | | | |
| 3. KETO- | 70% PVA | 30% | Three- | Inner feed: |
| Suspension | 4-88 | | fluid | Ketoconazole |
| 30% DL; three- | | | nozzle | in Methanol; |
| fluid | | | | Outer feed: |
| | | | | PVA 4-88 in |
| | | | | water |

Two-fluid nozzle (i): PVA 4-88 is dissolved in deionized water on a magnetic stirrer with heating at a rotation speed of 200 rpm at 60° C. Ketoconazole is dissolved in the respective Ethanol volume and added to the PVA solution. The solution (Formulation No. 1) or suspension (Formulation No. 2) is fed into the Büchi Spray-dryer. The suspension is stirred during the spraying process while the solution is kept without stirring to avoid any sediment formed during the process to be drawn into the spraying chamber.

Three-fluid nozzle (ii): PVA 4-88 solution in water is prepared as described above (Formulation No. 3). Ketoconazole is dissolved in Methanol (MeOH). The feed solutions are drawn up with a 50 mL syringe and inserted into the PHD ULTRA™ Syringe Pump (Harvard Apparatus, US) with PVA 4-88 in water in the outer feed and Ketoconaozole in MeOH in the inner feed.

The following conditions were applied for the spray-drying process: inlet temperature of 100° C., outlet temperature of 60° C., drying air flow rate of 35 m³/h (Aspirator: 100%) and an atomization air flow rate of 670 L/min (N2: 55 mm). The feed solution flow rate is adjusted to the outlet temperature.

### PXRD

Diffractograms of powders were measured using an Miniflex 600 X-ray diffractometer (Rigaku, Japan) with CuKα radiation (λ=1.54 A). Samples were scanned in reflectance mode from 3° to 50° 2θ (deg), with a scan speed of 10° 2θ (deg)/min and a step size of 0.020° 2θ (deg) and results are shown in FIG. 2. The acceleration voltage and current are 45 kV and 15 mA.

### DSC

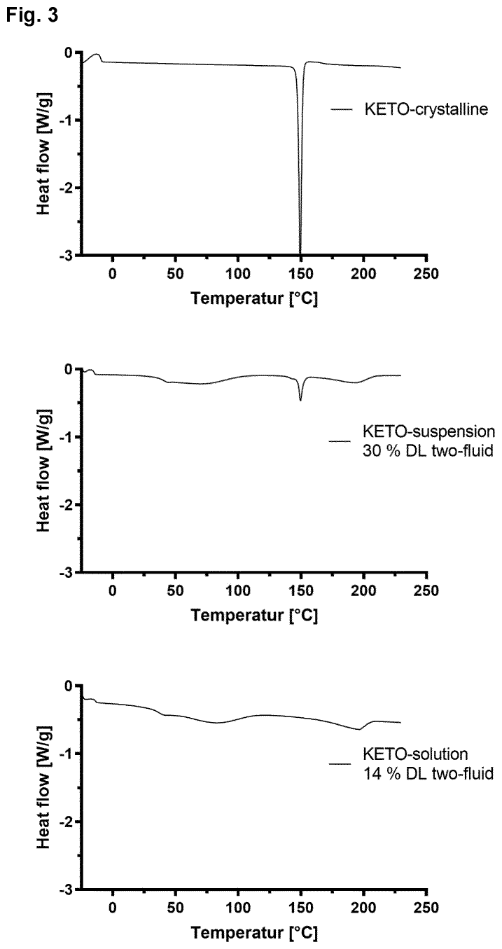

The DSC thermograms of the samples as shown in FIG. 3 were collected using a DSC 3 (METTLER, U.S.) under a nitrogen gas flow of 50 ml/min. A total of 2-5 mg sample powder was filled into an aluminum Tzero pan (40 μL) and sealed with an aluminum Tzero lid. The sample was analyzed at a heating rate of 5° C./min from −25° C. to 230° C.

As seen in the FIGS. 2 and 3, spray-drying of a suspension of ketoconazole with PVA 4-88 leads to a partly crystalline product while spraying of the respective solution leads to an amorphous product.

In both examples a two-fluid nozzle set-up is used. It can be seen that a drug loading of 14% leads to a fully amorphous product, whereas that is not possible with a drug loading of 30% when using a two-fluid nozzle.

The sharp signals in the x-ray diffraction (FIG. 2) of the crystalline Ketoconazole can be found at 7.1, 17.4, 23.6 and 27.4° 2θ. These peaks are still visible as predominant signals in the spray-dried suspension of KETO in 4-88 PVA while these patterns are not detectable in the XRD of the spray-dried product using the respective solution. The melting point for ketoconazole is defined to be in the range of 148° C.-150° C. and can be identified in the DSC (FIG. 3) of the ketoconazole-PVA suspension whereas in the DSC curve of the spray-dried ketoconazole-PVA solution no such peak can be identified.

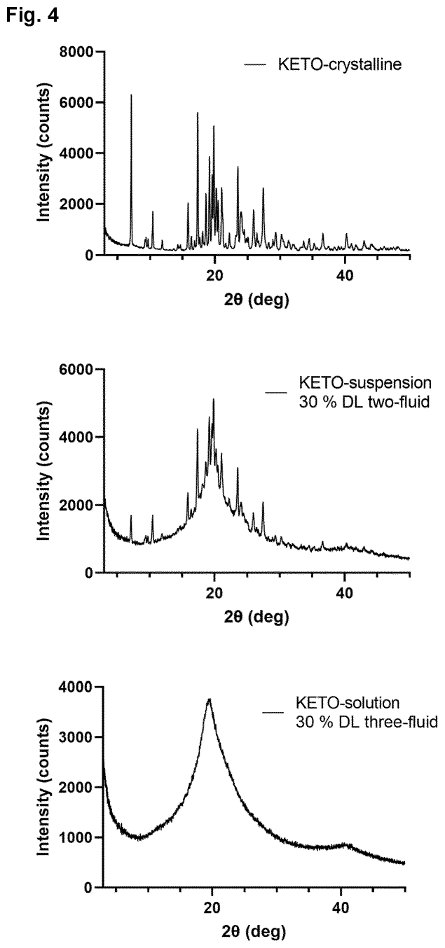

Increasing the drug loading to a desired amount of 30% DL is not feasible for KETO in the used solvent system as a suspension is obtained with DL of >14%. To higher the DL, a three-fluid nozzle set-up is used. FIG. 4 shows the comparison of 30% DL of KETO with 4-88 PVA using a two-fluid nozzle and suspension vs. a three-fluid nozzle and solution set-up.

No sharp peaks at 7.1, 17.4, 23.6 and 27.4° 2θ can be detected for the three-fluid nozzle set-up.

### Example 2: Dissolution of ASDs with Different APIs and PVA Grades

Dissolution was tested using three-fluid nozzle spray-dried ASDs with indomethacine (INDO), ritonavir (RITO) and ketoconazole (KETO) as APIs and PVA grades PVA 2-98, PVA 3-82, PVA 4-88 and PVA 5-74. In addition, PVA grades PVA 3-98 and 4-98 were investigated but the process of spray-drying this PVA grade was not feasible due to dissolving issues when preparing spray solutions.

### Spray-Drying Indomethacine ASDs

The following feed solutions were spray-dried using the Büchi spray-dryer B-290 (BÜCHI, Switzerland) equipped with a high-performance cyclone and the Büchi Inert Loop B-295 (BÜCHI, Switzerland), to inert the system with nitrogen.

Due to the different solubilities, a three fluid nozzle was used. Feed 1, the inner phase, contains 3 g indomethacin in a mixture of 50 mL of acetone and deionized water at a volume ratio of 70 to 30. Feed 2, the outer phase, contains the polymer solution. 7 g of PVA 5-74, PVA 3-82, PVA 4-88, PVA 2-98, PVA 3-98, and PVA 4-98 are solved in 50 mL of deionized water on a magnetic stirrer with heating at a rotation speed of 200 rpm at 60° C. The feed solutions are drawn up with a 50 mL syringe and inserted into the PHD ULTRA™ Syringe Pump (Harvard Apparatus, US).

The following conditions were applied for the spray-drying process: inlet temperature of 90° C., outlet temperature of 50° C., drying air flow rate of 35 m3/h (Aspirator: 100%) and an atomization air flow rate of 670 L/min (N2: 55 mm). The feed solution flow rate is adjusted to the outlet temperature.

### Drug Content Determination of Indomethacine Via RP-HPLC

To determine drug loading, one gram of sample is weighed in a 250 mL volumetric flask. 125 mL of Milli-Q-water is added and stirred for 45 min at 500 rpm on a magnetic stirring bar. After filling up the solution with acetonitrile to 250 mL, the sample must be filtered through a 0.45 μm filter and diluted 1 to 5 with mobile phase.

The mobile phase is a mixture of 1000 mL of acetonitrile and 1000 mL of indomethacin buffer USP (0.01M NaH₂PO*H₂O 1.38 g/L+0.01M Na₂HPO₄ 1.41 g/L) and is placed in the ultrasonic bath for 15 min.

An Agilent 1260 infinity HPLC system (Agilent, Santa Clara, USA) equipped with an Agilent 1260 II Variable Wavelength Detector was used. A LC-18 (Supelco, 4.0×300 mm, 5 μm) column was used for the quantification with a column oven temperature of 40° C. The injection volume was 10 μl and the flow rate was 1 ml/min. The UV detection wavelength was 254 nm and the retention time was approx. 2.4 min. A twofold determination is to be performed and double injection.

### Dissolution Measurement Indomethacine ASDs

The dissolution of indomethacin is executed on the Dissolution Sotax AT7 smart (Sotax AG, Lörrach, Deutschland) which is equipped with a fraction collector (Sotax C613) and a UV-visible spectrophotometer (UV-VIS Agilent 8453).

Sample (25 mg of IND or 83.3 mg of solid dispersion particles (30% drug loading) was added to 900 mL of SGFsp for a disintegration test with paddle stirring at a rotation speed of 75 rpm at 37° C. The drug concentration in the medium was measured spectrophotometrically at 318 nm (UV-VIS Agilent 8453). The pathlength is 10 mm.

The solubility enhancement was investigated using simulated gastric fluid (SGF) as Indomethacin is poorly soluble in acidic pH. FIG. 5 shows the dissolution enhancement with spray-dried ASDs prepared with PVA 4-88, PVA 3-82, PVA 2-98, and PVA 5-74. IND-PVA 3-82 shows superior dissolution values, whereas IND-PVA 4-88 and IND-PVA 5-74 display similar dissolution behavior. IND-PVA 2-98 gave a sufficient solution when dissolved and was applicable in the spray-drying process but the product shows an unfavorable dissolution behavior. Additionally, PVA 4-98 and 3-98 were investigated, but the process of spay drying both PVA grades was not feasible due to dissolving issues when preparing spray solutions.

### Spray-Drying Ritonavir ASDs

The following feed solutions were spray-dried using the Büchi spray-dryer B-290 (BÜCHI, Switzerland) equipped with a high-performance cyclone and the Büchi Inert Loop B-295 (BÜCHI, Switzerland), to inert the system with nitrogen.

Due to the different solubilities, a three fluid nozzle was used. Feed 1, the inner phase, contains 3 g ritonavir in 50 mL of ethanol. Feed 2, the outer phase, contains the polymer solution. 7 g of PVA 4-88 or PVA 3-82 are solved in 50 mL of deionized water on a magnetic stirrer with heating at a rotation speed of 200 rpm at 60° C. The feed solutions are drawn up with a 50 mL syringe and inserted into the PHD ULTRA™ Syringe Pump (Harvard Apparatus, US).

The following conditions were applied for the spray-drying process: inlet temperature of 100° C., outlet temperature of 60° C., drying air flow rate of 35 m³/h (Aspirator: 100%) and an atomization air flow rate of 670 L/min (N2: 55 mm). The feed solution flow rate is adjusted to the outlet temperature.

### Drug Content Determination of Ritonavir Via RP-HPLC

Five to ten milligram is weighed in a 50 mL volumetric flask. 25 mL of Mobile Phase is added and stirred at 500 rpm on a magnetic stirring bar until a clear solution is given. After filling up the solution with mobile phase, the sample must be filtered through a 0.45 μm filter.

An Agilent 1260 infinity HPLC system (Agilent, Santa Clara, USA) equipped with an Agilent 1260 II Variable Wavelength Detector was used. A RP-C18 (Supelco, 4.6×150 mm, 5 μm) column was used for the quantification. The injection volume was 20 μl and the flow rate was 1 ml/min.

The mobile phase was a mixture of 2 g/l potassium phosphate monobasic in water and acetonitrile at a volume ratio of 45 to 55. The mobile phase was adjusted to pH of 4.0±0.05 using phosphoric acid. The UV detection wavelength was 215 nm.

### Dissolution Measurement Ritonavir ASDs

The dissolution of the samples was determined in fasted state simulated intestinal fluid V2 (FaSSIF V2, Biorelevant). 20 mg of bulk drug or the equivalent of raw material were weighted and placed into a 100 ml of Erlenmeyer flask, 20 ml of dissolution medium was added and stirred at 200 rpm using a magnetic stirring bar.

2 ml of dissolution medium were withdrawn from the dissolution vessels at predetermined time points and immediately replaced by 2 ml of fresh dissolution medium (5, 10, 20, 40, 60, 90, 120 min). The samples were then filtered through a 0.45 μm filter, diluted using acetonitrile, and subsequently filtered again through a 0.45 μm filter. The drug content of the samples was analyzed using RP-HPLC.

An Agilent 1260 infinity HPLC system (Agilent, Santa Clara, USA) equipped with an Agilent 1260 II Variable Wavelength Detector was used. A RP-C18 (Supelco, 4.6×150 mm, 5 μm) column was used for the quantification.

The mobile phase contains a mixture of 2 g/l potassium phosphate monobasic in water and acetonitrile at a volume ratio of 45 to 55. The mobile phase was adjusted to pH of 4.0±0.05 using phosphoric acid. The UV detection wavelength was 215 nm at a flow rate of 1 ml/min and an injection volume of 20 μl.

Ritonavir displays low solubility and bioavailability values and is therefore classified in the BSC class IV. The solubility of the spray-dried amorphous solution of Ritonavir is enhanced when using PVA as seen in FIG. 6. To show dissolution improvement, the biorelevant media FaSSIF (fasted state simulated intestinal fluid) was used which imitates the process of API dissolving in the upper intestine.

PVA 4-88 improves dissolution of the crystalline API to about four orders in magnitude. PVA 3-82 performs superior and shows improvement of dissolution of seven orders of magnitude compared to the crystalline substance at 120 minutes.

### Spray-Drying Ketoconazole ASDs

The following feed solutions were spray-dried using the Büchi spray-dryer B-290 (BÜCHI, Switzerland) equipped with a high-performance cyclone and the Büchi Inert Loop B-295 (BÜCHI, Switzerland), to inert the system with nitrogen.

Due to the different solubilities, a three fluid nozzle was used. Feed 1, the inner phase, contains 3 g ketoconazole in 50 mL of ethanol. Feed 2, the outer phase, contains the polymer solution. 7 g of PVA 2-98, PVA 3-82, PVA 4-88 and PVA 5-74 are solved in 50 mL of deionized water on a magnetic stirrer with heating at a rotation speed of 200 rpm at 60° C. The feed solutions are drawn up with a 50 mL syringe and inserted into the PHD ULTRA™ Syringe Pump (Harvard Apparatus, US).

The following conditions were applied for the spray-drying process: inlet temperature of 100° C., outlet temperature of 60° C., drying air flow rate of 35 m³/h (Aspirator: 100%) and an atomization air flow rate of 670 L/min (N2: 55 mm). The feed solution flow rate is adjusted to the outlet temperature.

### Dissolution Measurement Ketoconazole ASDs

The dissolution of ketoconazole is executed on the Dissolution Sotax AT7 smart (Sotax AG, Lörrach, Deutschland) which is equipped with a fraction collector (Sotax C613) and a UV-visible spectrophotometer (UV-VIS specord 200 plus).

Sample (400 mg of ketoconazole or 1333 mg of solid dispersion particles (30% drug loading)) was added to 500 mL of FaSSIF for a disintegration test with paddle stirring at a rotation speed of 50 rpm at 37° C.

Samples of 2 ml are taken after 30, 60, 120, 135, 150, 180, 240 and 300 min. Sample solutions are filtered through a 0.45 μm filter into a vial and diluted at an 1 to 1 ratio with ethanol.

An Agilent 1260 infinity HPLC system (Agilent, Santa Clara, USA) equipped with an Agilent 1260 II Variable Wavelength Detector was used. A LC-18 (Supelco, 4.0×300 mm, 5 μm) column was used for the quantification with a column oven temperature of 40° C. The injection volume was 5 μl and the flow rate was 2 ml/min. The mobile phase is divided into two eluents (70% Eluent A/30% Eluent B). Eluent A is a mixture of 10 ml Diisopropylamin in 5 L methanol. For Eluent B, 5 g/L ammonium acetate in milli-Q-water is used.

The UV detection wavelength was 225 nm and the retention time was approx. 5.7 min.

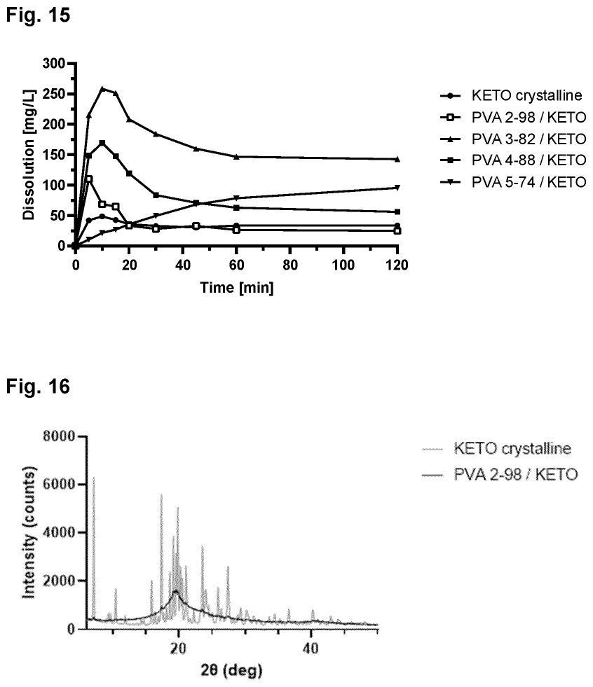

FIG. 15 shows the enhanced dissolution of 30% (w/w) ketoconazole as a spray dried ASD containing PVA 4-88, PVA 3-82, PVA 5-74 and PVA 2-98 in FaSSIF compared to the crystalline product. PVA 3-82, followed by PVA 4-88 enhance dissolution of Ketoconazole significantly. PVA 2-98 shows an enhanced release within the first 20 minutes, but recrystallization of Ketoconazole leads to dissolution comparable to the crystalline substance. PVA 5-74 shows an almost linear increase in dissolution of Ketoconazole reaching second best dissolution values at the 120 time point.

### Example 3: XRDs of ASDs with Different APIs and PVA Grades

XRD measurement was performed using three-fluid nozzle spray-dried ASDs with ketoconazole (KETO), ritonavir (RITO) and indomethacine (INDO) as APIs and PVA grades PVA 2-98, PVA 3-82, PVA 4-88 and PVA 5-74.

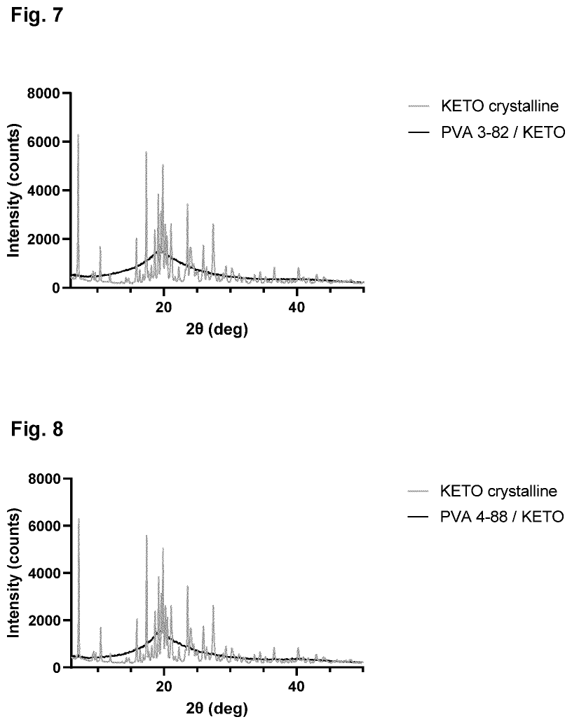

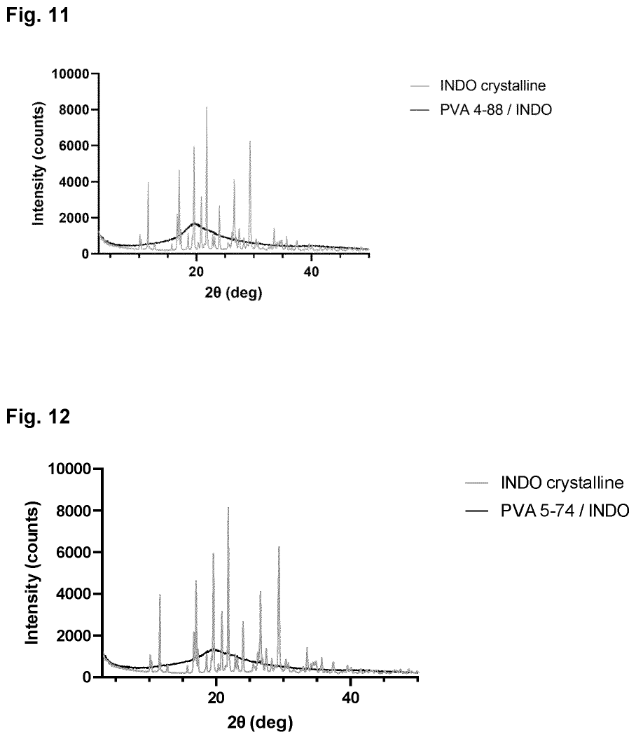

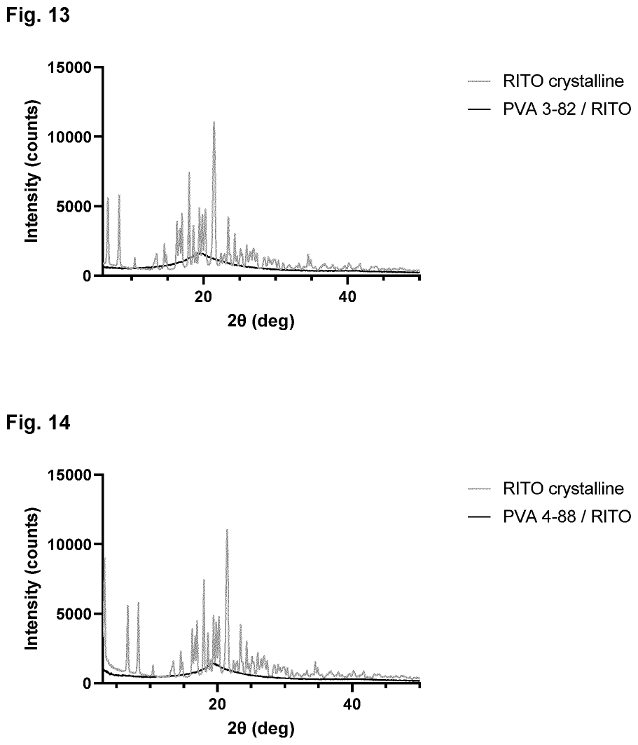

Spray-drying of ASDs has been prepared as described in Example 2, XRD measurements have been performed as described in Example 1. Results are shown in FIG. 7 to 14.

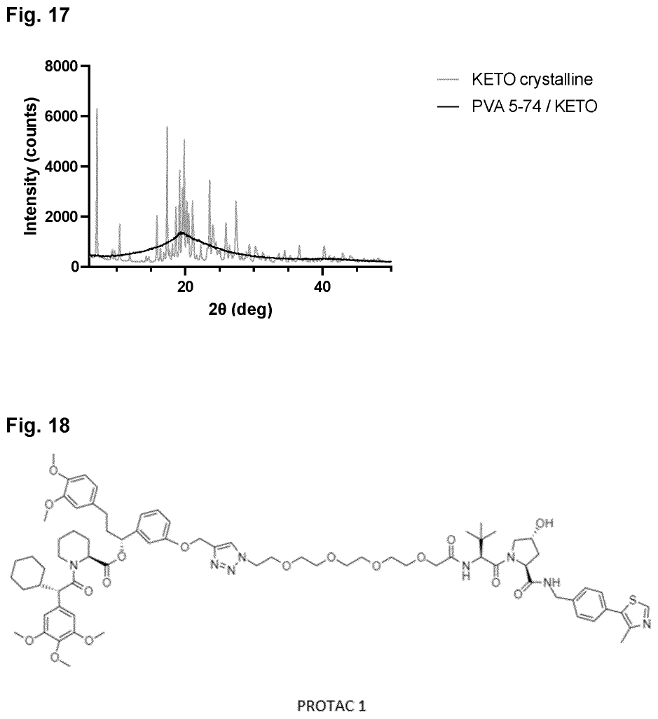

XRD measurements (FIGS. 7 to 14, 16 and 17) show that the spray-drying with PVA 3-82, 4-88 and 5-74 leads to products with a good amorphization of the API. The XRDs of the spray-dried PVA 2-98 (FIGS. 9 and 16) show, that the products do not have a complete amorphous state adding to the lower dissolution rate (FIG. 5).

### Example 4: Three-Fluid Nozzle Spray-Drying of ASDs with PROTACs as API

### Spray-Drying PROTAC ASDs

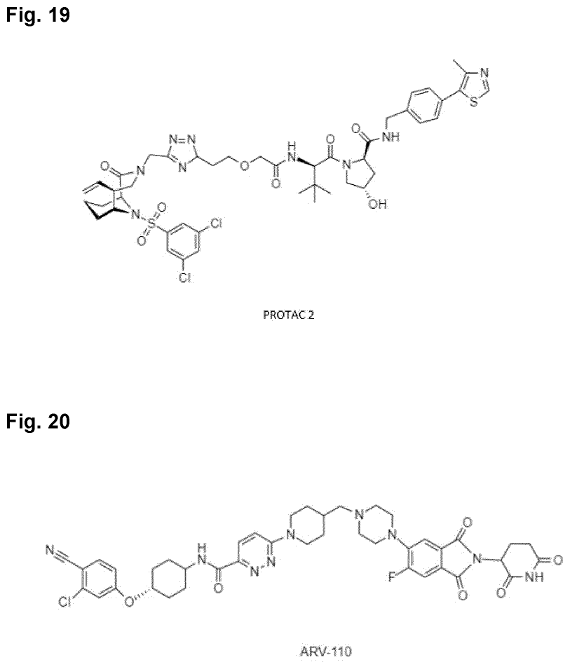

The following PROTACs were used: PROTAC 1 (molecular formula see FIG. 18), PROTAC 2 (molecular formula see FIG. 19) and PROTAC ARV-110 (molecular formula see FIG. 20).

For PROTACs 1 and 2, the following feed solutions were spray dried using the Büchi spray dryer B-290 (BÜCHI, Switzerland) equipped with a high-performance cyclone and the Büchi Inert Loop B-295 (BÜCHI, Switzerland), to inert the system with nitrogen.

Due to the different solubilities, a three fluid nozzle was used. Feed 1, the inner phase, contains 70 mg of PROTAC in 25 mL of ethanol. Feed 2, the outer phase, contains the polymer solution. 140 mg of PVA 3-82 are dissolved in 25 mL of deionized water on a magnetic stirrer with heating at a rotation speed of 200 rpm at 60° C. The feed solutions are pumped into the spray dryer with the help of a peristaltic pump.

The following conditions were applied for the spray drying process: inlet temperature of 90° C., outlet temperature of 50° C., drying air flow rate of 35 m3/h (Aspirator: 100%) and an atomization air flow rate of 670 L/min (N2: 55 mm). The feed solution flow rate is adjusted to the outlet temperature.

For PROTAC ARV-110, the following feed solutions were spray dried using the Büchi spray dryer B-290 (BÜCHI, Switzerland) equipped with a high-performance cyclone and the Büchi Inert Loop B-295 (BÜCHI, Switzerland), to inert the system with nitrogen.

Due to the different solubilities, a three fluid nozzle was used. Feed 1, the inner phase, contains 400 mg of PROTAC in 550 mL of DCM:MeOH (40:60). Feed 2, the outer phase, contains the polymer solution. 932 mg of PVA 3-82 are dissolved in 550 mL of deionized water on a magnetic stirrer. The feed solutions are pumped into the spray dryer with the help of a peristaltic pump.

The following conditions were applied for the spray drying process: inlet temperature of 80° C., outlet temperature of 45° C., drying air flow rate of 35 m3/h (Aspirator: 100%) and an atomization air flow rate of 670 L/min (N2: 55 mm). The feed solution flow rate is adjusted to the outlet temperature.

### Drug Content Determination of PROTACs

For PROTACs 1 and 2, 1 mg sample is dissolved in 50 ml of DMSO. The sample has to be filtered through a 0.45 μm filter.

An Agilent 1260 infinity HPLC system (Agilent, Santa Clara, USA) equipped with an Agilent 1260 II Variable Wavelength Detector was used. A RP-C18 (Phenomenex, 4.6×250 mm, 5 μm) column was used for the quantification. The injection volume was 50 μl and the flow rate was 1 ml/min.

The eluents are composed as follows: Eluent A: 0.1% TFA in water; Eluent B: 0.1% TFA in acetonitrile.

For PROTAC ARV-110, 1 mg sample is dissolved in 50 ml of DMSO. The sample has to be filtered through a 0.45 μm filter.

An Agilent 1260 infinity HPLC system (Agilent, Santa Clara, USA) equipped with an Agilent 1260 II Diode-Array Detector was used. A C8-Column (Waters XBridge Column C8, 4.6×50 mm, 3.5 μm) column was used for the quantification. The injection volume was 5 μl and the flow rate was 1.7 ml/min. The eluents are composed as follows: Eluent A: 0.1% formic acid in water; Eluent B: 0.1% formic acid in acetonitrile.

### Dissolution Measurement of PROTAC ASDs

For PROTACs 1 and 2, the dissolution of the samples was determined at room temperature in phosphate buffer pH 6.8 as dissolution medium. 5 mg of bulk drug or spray dried powder samples equivalent to 5 mg of drug were weighted and placed into a 100 ml of Erlenmeyer flask, subsequently a magnetic stirring bar was added to the Erlenmeyer flask and stirred at 200 rpm, then 25 ml of dissolution medium was added. (concentration: 0.2 mg/ml)

At predetermined time points (5, 10, 20, 40, 60, 90, 120 min), 500 μl of sample were withdrawn from the dissolution vessels and immediately replaced by 500 μl of fresh dissolution medium. The dissolution samples were then filtered through a 0.45 μm filter. Finally, the samples were analyzed towards drug content using HPLC.

An Agilent 1260 infinity HPLC system (Agilent, Santa Clara, USA) equipped with an Agilent 1260 II Variable Wavelength Detector was used. A RP-C18 (Phenomenex, 4.6×250 mm, 5 μm) column was used for the quantification. The injection volume was 50 μl and the flow rate was 1 ml/min.

The eluents are composed as follows: Eluent A: 0.1% TFA in water; Eluent B: 0.1% TFA in acetonitrile. The method was as followed: linear gradient of 0-100% Eluent B in 25 minutes.

For PROTAC ARV-110, the dissolution of the samples was determined at room temperature in phosphate buffer pH 6.8 as dissolution medium. 5 mg of bulk drug or spray dried powder samples equivalent to 5 mg of drug were weighted and placed into a 100 ml of Erlenmeyer flask, subsequently a magnetic stirring bar was added to the Erlenmeyer flask and stirred at 200 rpm, then 25 ml of dissolution medium was added. (concentration: 0.2 mg/ml)

At predetermined time points (5, 10, 20, 40, 60, 90, 120 min), 500 μl of sample were withdrawn from the dissolution vessels and immediately replaced by 500 μl of fresh dissolution medium. The dissolution samples were then filtered through a 0.45 μm filter. The sample is diluted with Eluent B (1:1). Finally, the samples were analyzed towards drug content using HPLC.

An Agilent 1260 infinity HPLC system (Agilent, Santa Clara, USA) equipped with an Agilent 1260 II Variable Wavelength Detector was used. A C8-Column (Waters XBridge Column C8, 4.6×50 mm, 3.5 μm) column was used for the quantification. The injection volume was 5 μl and the flow rate was 1.7 ml/min.

The eluents are composed as follows: Eluent A: 0.1% formic acid in water; Eluent B: 0.1% formic acid in acetonitrile. Method: linear gradient of 10% to 90% Eluent B in 2 minutes at 1.7 ml/min flow rate.

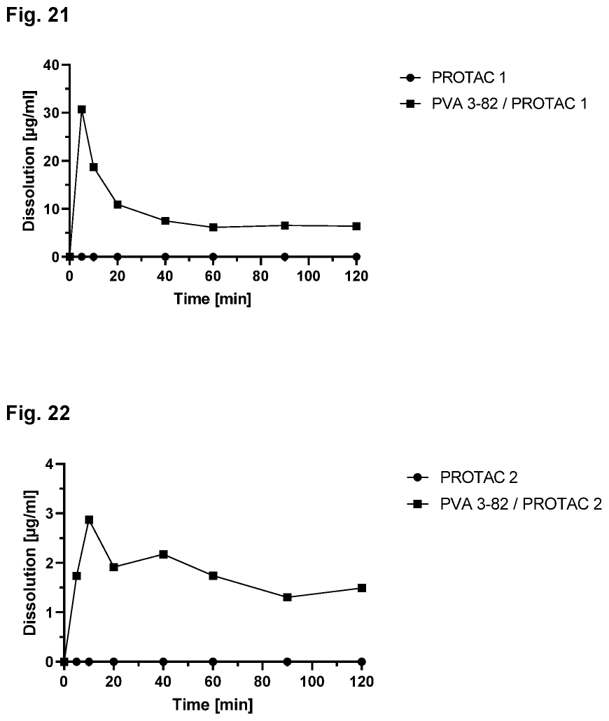

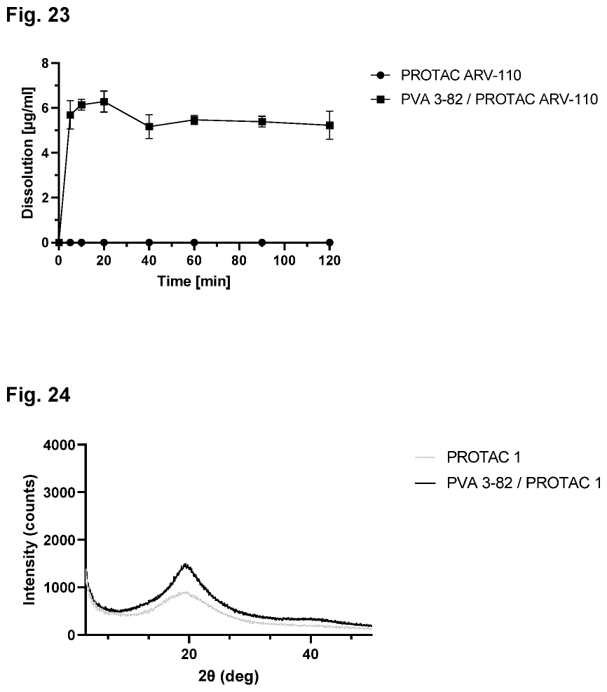

FIG. 21 to 23 show dissolution of the spray dried dispersion of PROTAC 1, PROTAC 2 and ARV 110 with PVA 3-82 compared to the raw product PROTAC 1, PROTAC 2 and ARV110 in phosphate buffer at pH 6.8. It can be seen that neither the raw PROTAC 1, PROTAC 2 nor ARV110 show any measurable dissolution over the time of 120 minutes in phosphate buffer. For all three examples, the spray dried dispersion of PVA 3-82 with the respective PROTACs show enhances dissolution. For PROTAC 1, dissolution is significantly increased within the first 30 minutes with a continuous decrease of release values whereas for PROTAC 2 the enhanced release is stabilized over the period of 120 minutes. Additionally, ARV110 is stabilized at a high level of dissolved drug compared to the crystalline substance, showing a fast onset of the spray dried dispersion as well as a prolonged release over the complete time period of 120 minutes.

### X-Ray Powder Diffraction

Diffractograms of powders were measured using an Miniflex 600 X-ray diffractometer (Rigaku, Japan) with CuKα radiation (λ=1.54 A). Samples were scanned in reflectance mode from 3° to 50° 2θ (deg), with a scan speed of 10° 2θ (deg)/min and a step size of 0.020° 2θ (deg). The acceleration voltage and current are 45 kV and 15 mA.

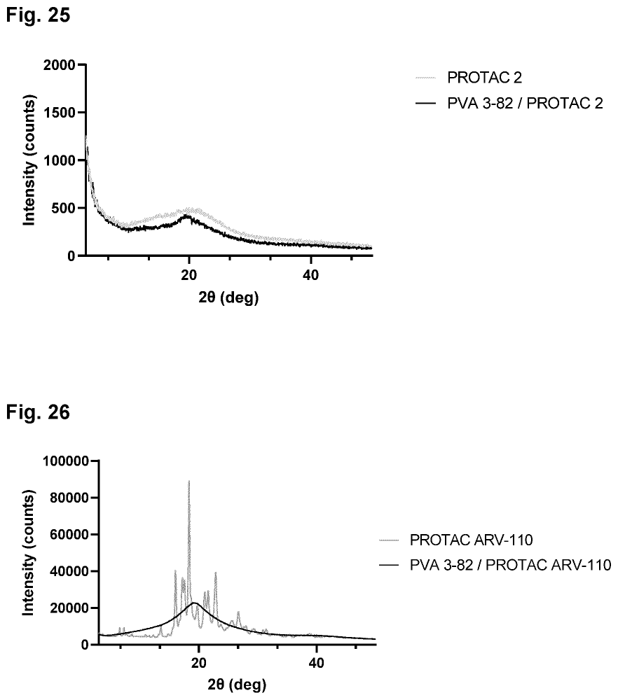

XRD measurements (FIG. 24 to 25) show that both, the raw material and the spray dried PROTAC 1 and PROTAC are amorphous. For the PROTAC ARV-110 it can be seen that the raw material is crystalline and the spray dried dispersion with PVA 3-82 is amorphous (FIG. 26).

### Example 5: Stability Measurements of ASDs

To investigate the stability of the PVA 3-82-ARV110 formulation, the spray dried product and raw product were stored using the following conditions and investigated in dissolution experiments and x-ray diffraction after 4 weeks.

| Storage conditions | Time | Analytics |
|---|---|---|
| Fridge, 5° C. | 4 weeks | Dissolution measurement |
| | | X-ray powder diffraction |
| 25° C. at 60% rH | 4 weeks | Dissolution measurement |
| | | X-ray powder diffraction |

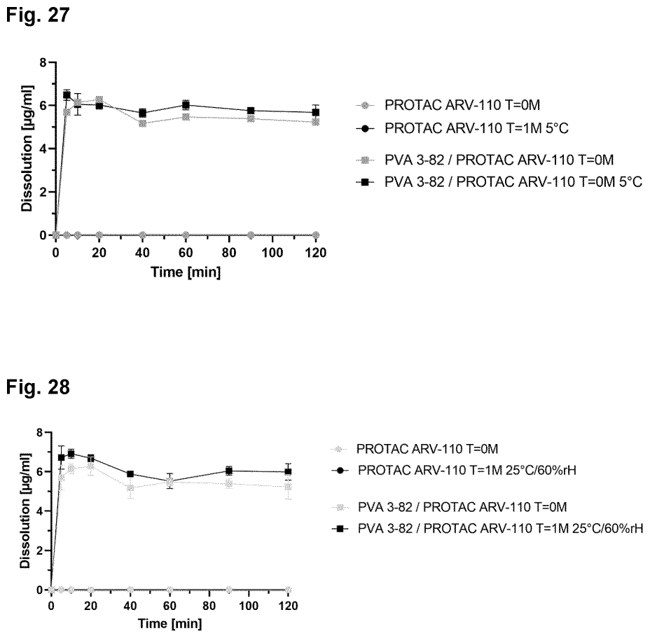

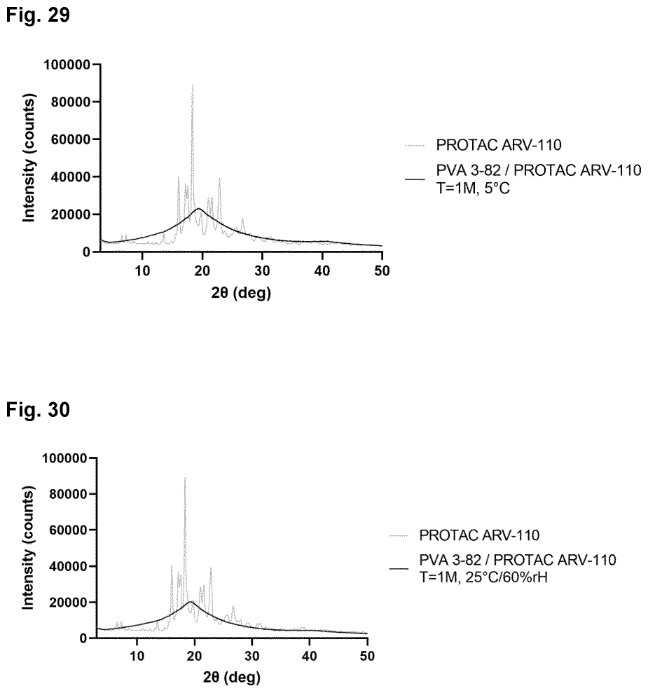

The spray dried PVA 3-82/ARV110 samples are still amorphous after 4 weeks stored at either 5° C. in the fridge (FIG. 29) and 25° C. at 60% rH (FIG. 30). Additionally, the samples show no significant reduction in dissolution enhancement when comparing to the dissolution done at t=0 (FIGS. 27 and 28).

Dissolution Measurement of PVA 3-82/ARV110 from Stability Studies

For PROTAC ARV-110, the dissolution of the samples was determined at room temperature in phosphate buffer pH 6.8 as dissolution medium. 5 mg of bulk drug or spray dried powder from the stability studies equivalent to 5 mg of drug were weighted and placed into a 100 ml of Erlenmeyer flask, subsequently a magnetic stirring bar was added to the Erlenmeyer flask and stirred at 200 rpm, then 25 ml of dissolution medium was added. (concentration: 0.2 mg/ml)

At predetermined time points (5, 10, 20, 40, 60, 90, 120 min), 500 μl of sample were withdrawn from the dissolution vessels and immediately replaced by 500 μl of fresh dissolution medium. The dissolution samples were then filtered through a 0.45 μm filter. The sample is diluted with Eluent B (1:1). Finally, the samples were analyzed towards drug content using HPLC.

An Agilent 1260 infinity HPLC system (Agilent, Santa Clara, USA) equipped with an Agilent 1260 II Variable Wavelength Detector was used. A C8-Column (Waters XBridge Column C8, 4.6×50 mm, 3.5 μm) column was used for the quantification. The injection volume was 5 μl and the flow rate was 1.7 ml/min.

The eluents are composed as follows: Eluent A: 0.1% formic acid in water; Eluent B: 0.1% formic acid in acetonitrile. Method: linear gradient of 10% to 90% Eluent B in 2 minutes at 1.7 ml/min flow rate.

### X-Ray Powder Diffraction

Diffractograms of powders were measured using an Miniflex 600 X-ray diffractometer (Rigaku, Japan) with CuKα radiation (λ=1.54 A). Samples were scanned in reflectance mode from 3° to 50° 2θ (deg), with a scan speed of 10° 2θ (deg)/min and a step size of 0.020° 2θ (deg). The acceleration voltage and current are 45 kV and 15 mA.

### REFERENCES

Bhujbal, S. V., B. Mitra, U. Jain, Y. Gong, A. Agrawal, S. Karki, L. S. Taylor, S. Kumar, and Q. Tony Zhou. 2021. ‘Pharmaceutical amorphous solid dispersion: A review of manufacturing strategies’, Acta Pharm Sin B, 11: 2505-36.
Bhujbal, S. V., Y. Su, V. Pathak, D. Y. Zemlyanov, A. A. Cavallaro, E. J. Munson, L. S. Taylor, and Q. T. Zhou. 2021. ‘Effect of Storage Humidity on Physical Stability of Spray-Dried Naproxen Amorphous Solid Dispersions with Polyvinylpyrrolidone: Two Fluid Nozzle vs. Three Fluid Nozzle’, Pharmaceutics, 13.
Brough et al., Use of Polyvinyl Alcohol as a Solubility Enhancing Polymer for Poorly Water-Soluble Drug Delivery (Part 1), AAPS PharmSciTech Vol. 17, No. 1, p. 176 (Jan. 2, 2016)
Focaroli, S., G. Jiang, P. O'Connell, J. V. Fahy, and A. M. Healy. 2020. ‘The Use of a Three-Fluid Atomising Nozzle in the Production of Spray-Dried Theophylline/Salbutamol Sulphate Powders Intended for Pulmonary Delivery’, Pharmaceutics, 12.
Fouad, E. A., M. EI-Badry, G. M. Mahrous, F. K. Alanazi, S. H. Neau, and I. A. Alsarra. 2011. ‘The use of spray-drying to enhance celecoxib solubility’, Drug Dev Ind Pharm, 37:1463-72.
Hugo, M., K. Kunath, and J. Dressman. 2013. ‘Selection of excipient, solvent and packaging to optimize the performance of spray-dried formulations: case example fenofibrate’, Drug Dev Ind Pharm, 39: 402-12.
Kim, D. H., Y. W. Kim, Y. Y. Tin, M. T. Soe, B. H. Ko, S. J. Park, and J. W. Lee. 2021. ‘Recent Technologies for Amorphization of Poorly Water-Soluble Drugs’, Pharmaceutics, 13.
Ziaee, A., A. B. Albadarin, L. Padrela, T. Femmer, E. O'Reilly, and G. Walker. 2019. ‘Spray-drying of pharmaceuticals and biopharmaceuticals: Critical parameters and experimental process optimization approaches’, Eur J Pharm Sci, 127: 300-18.

1. An amorphous solid dispersion comprising at least one active pharmaceutical ingredient in a polyvinyl alcohol matrix, wherein the amorphous solid dispersion is obtained by a spray-drying process using polyvinyl alcohol having a hydrolysis degree of 90% or lower.

2. The amorphous solid dispersion according to claim 1, wherein the polyvinyl alcohol has a hydrolysis degree of between 74% to 88% and a viscosity of a 4% solution at 20° C. of between 2 to 5 mPas.

3. The amorphous solid dispersion according to claim 1, wherein the polyvinyl alcohol is PVA 3-80, PVA 3-81, PVA 3-82 PVA 3-83, PVA 2-88, PVA 3-88, PVA 4-88, PVA 5-88, PVA 2-74, PVA 3-74, PVA 4-74 or PVA 5-74.

4. The amorphous solid dispersion according to claim 1, wherein the polyvinyl alcohol is PVA 3-82, 4-88 or 5-74.

5. The amorphous solid dispersion according to claim 1, wherein the polyvinyl alcohol is PVA 3-82.

6. The amorphous solid dispersion according to claim 1, wherein the active pharmaceutical ingredient is a PROTAC.

7. The amorphous solid dispersion according to claim 1, wherein the spray-drying process uses a three-fluid nozzle spray-drying.

8. Process for producing an amorphous solid dispersion of at least one active pharmaceutical ingredient in a polyvinyl alcohol matrix by spray-drying, comprising the steps of:
a) preparing a feed solution comprising the at least one active pharmaceutical ingredient and at least one polyvinyl alcohol having a hydrolysis degree of 90% or lower, and
b) spray-drying the feed solution.

9. Process for producing an amorphous solid dispersion of at least one active pharmaceutical ingredient in a polyvinyl alcohol matrix by three-fluid nozzle spray-drying, comprising the steps of:
a) preparing a first feed solution comprising the at least one active pharmaceutical ingredient,
b) preparing a second feed solution comprising a polyvinyl alcohol having a hydrolysis degree of 90% or lower, and
c) spray-drying the first and second feed solution with a three-fluid nozzle.

10. Process according to claim 9, wherein the polyvinyl alcohol has a hydrolysis degree of between 74% to 88% and a viscosity of a 4% solution at 20° C. of between 2 to 5 mPas.

11. Process according to claim 9, wherein the polyvinyl alcohol is PVA 3-82, 4-88 or 5-74.

12. Process according to claim 9, wherein the polyvinyl alcohol is PVA 3-82.

13. Process according to claim 9, wherein the active pharmaceutical ingredient is a PROTAC.

14. Amorphous solid dispersion of at least one active pharmaceutical ingredient in a polyvinyl alcohol matrix obtainable by a process according to claim 9.

15. Amorphous solid dispersion according to claim 14, wherein the active pharmaceutical ingredient is a PROTAC.

16. Process according to claim 8, wherein the polyvinyl alcohol has a hydrolysis degree of between 74% to 88% and a viscosity of a 4% solution at 20° C. of between 2 to 5 mPas.

17. Process according to claim 8, wherein the polyvinyl alcohol is PVA 3-82, 4-88 or 5-74.

18. Process according to claim 8, wherein the polyvinyl alcohol is PVA 3-82.

19. Process according to claim 8, wherein the active pharmaceutical ingredient is a PROTAC.

20. Amorphous solid dispersion of at least one active pharmaceutical ingredient in a polyvinyl alcohol matrix obtainable by a process according to claim 8.
